SEQUENCE LISTING (12 sequences)

SEQ ID NO: 1 - DNA, 22 bp, Artificial sequence
  NKD Primer Forward
cagccagagcaagaggagcgtc

SEQ ID NO: 2 - DNA, 22 bp, Artificial sequence
  NKD primer reverse
ccggcgagatctaagtagtggt

SEQ ID NO: 3 - DNA, 22 bp, Artificial sequence
  NKG2E primer forward
gccagcattttaccttcctcat

SEQ ID NO: 4 - DNA, 22 bp, Artificial sequence
  NKG2E primer reverse
aacatgatgaaaccccgtctaa

SEQ ID NO: 5 - DNA, 19 bp, Artificial sequence
  CECR6 primer forward
acagtcggtgtggaatgtc

SEQ ID NO: 6 - DNA, 20 bp, Artificial sequence
  CECR6 primer reverse
agaagggagaaggggaaaca

SEQ ID NO: 7 - DNA, 22 bp, Artificial sequence
  CECR6 ChIP primer forward
tgataaatgctagtgaggtgcc

SEQ ID NO: 8 - DNA, 22 bp, Artificial sequence
  CECR6 ChIP primer  reverse
agaaccgcctgctcctaacaat

SEQ ID NO: 9 - DNA, 22 bp, Artificial sequence
  NKD ChIP primer forward
gggtcaggacgagtgttttctt

SEQ ID NO: 10 - DNA, 22 bp, Artificial sequence
  NKD ChIP primer reverse
accccggaccaaatttcagtta

SEQ ID NO: 11 - DNA, 20 bp, Artificial sequence
  NKG2E ChIP primer forward
agccacccaaagtctcctat

SEQ ID NO: 12 - DNA, 20 bp, Artificial sequence
  NKG2E ChIP primer reverse
ttcagtggagaggtcaggtt

(19) United States
(12) Patent Application Publication
(10) Pub. No.: US 2008/0319051 A1
(43) Pub. Date: Dec. 25, 2008

(54) LIQUIRITIGENIN AND DERIVATIVES AS SELECTIVE ESTROGEN RECEPTOR BETA AGONISTS

(75) Inventor: Isaac COHEN, Piedmont, CA (US)

Correspondence Address: WILSON SONSINI GOODRICH & ROSATI, 650 PAGE MILL ROAD, PALO ALTO, CA, 94304-1050, US

(73) Assignee: Bionovo, Inc., Emeryville, CA (US)

(21) Appl. No.: 11/767,380

(22) Filed: Jun. 22, 2007

(51) Int. Cl. A61K 31/352; A61P 13/00; A61P 25/00; A61P 29/00; A61P 35/00; A61P 37/00; A61P 43/00; A61P 9/00

(52) U.S. Cl. 514/456

(57) ABSTRACT

The disclosure provides compositions comprising liquiritigenin, or derivatives, or prodrugs, useful as estrogen receptor beta selective agonists. The disclosure also provides methods of treating menopausal symptoms, and estrogen-dependent disorders, with said compositions.

### STATEMENT REGARDING FEDERALLY SPONSORED RESEARCH

The U.S. Government has a paid-up license in this invention and the right in limited circumstances to require the patent owner to license others on reasonable terms as provided for by the terms of Grant No. AT002173 awarded by the National Institutes of Health National Center for Complementary and Alternative Medicine.

### FIELD OF THE INVENTION

The disclosure provides compositions comprising liquiritigenin, or derivatives, or prodrugs, useful as estrogen receptor beta selective agonists. The disclosure also provides methods of treating menopausal symptoms, and estrogen-dependent disorders, with said compositions.

### BACKGROUND OF THE INVENTION

Menopause is often associated with an array of symptoms, such as hot flashes, night sweats, mood changes, urogenital atrophy and loss of bone density that have traditionally been treated with hormone therapy (HT). In addition, increased risk of cardiovascular disease and osteoporosis occur with onset of menopause. HT has been used successfully to treat a variety of conditions, such as osteoporosis, increased risk of cardiovascular disease in post-menopausal women and climacteric symptoms, such as hot flashes, decreased libido and depression. However, HT with estradiol (E₂), either alone or in combination with progestin, can lead to undesirable effects. A recent Women's Health Initiative (WHI) study was abruptly halted when preliminary results showed that HRT was associated with a 35% increased risk of breast cancer. The WHI trial also found that HT with estrogen plus progestin increases a woman's risk not only of breast cancer, but also of heart disease, and dementia (Wassertheil-Smoller et al., JAMA 289:2673-2684 (2003), Chlebowski et al., Jama 289:3243-3253 (2003), Shumaker et al., Jama 289:2651-2662 (2003), Manson et al., N Engl J Med 349:523-534 (2003), Rossouw et al., Jama 288:321-333.(2002)). In addition, a second arm of the WHI found that using estrogen alone increased the risk of stroke and dementia (Anderson et al., Jama 291:1701-1712 (2004), Shumaker et al., Jama 291:2947-2958 (2004)). The adverse impact of HT on breast cancer and blood clots indicate that new strategies are needed to treat menopausal symptoms.

Alternative drugs to traditional HT could potentially include selective estrogen receptor modulators (SERMs), such as tamoxifen and raloxifene. Although current SERM therapy has some favorable effects, such as improved bone mineral density (Delmas et al., N Engl J Med 337:1641-1647 (1997), Love et al., N Engl J Med 326:852-856 (1992)) and the prevention of breast cancer, SERMs exacerbate hot flashes (Cranney, Drug Saf 28:721-730 (2005)). Other pharmacological options for hot flashes include antidepressant therapy using serotonin and norepinephrine reuptake inhibitors, as well as other neuro-modulators, such as gabapentin (Loprinzi et al., Mayo Clin Proc 77:1159-1163 (2002)). However, the overall benefit of these treatments is unclear considering their moderate efficacy (Evans et al., Obstet Gynecol 105:161-166 (2005)), potential significant side effects (Sicat et al., Pharmacotherapy 24:79-93 (2004), Loprinzi et al., Lancet 356:2059-2063 (2000)) and lack of benefits on other menopausal symptoms, such as vaginal atrophy and osteoporosis.

Botanical dietary supplements used in Traditional Chinese Medicine (TCM) are used by many patients to relieve their menopausal symptoms. It has been reported that about 25% of women use botanical extracts to treat menopausal symptoms (Upchurch et al., J Womens Health (Larchmt) 16:102-113). Despite compelling evidence that estrogens cause breast cancer, observational studies show that women in Asian countries have the lowest incidence of breast cancer even though they consume large quantities of plant estrogens (phytoestrogens). Likewise, Asian women report minimal symptoms during menopause and are far less prone to experience hot flashes at the time of cessation of ovarian function. These findings have encouraged many menopausal women in the United States to take phytoestrogens present in soybeans or herbal therapies as an alternative to estrogen, hoping to alleviate hot flashes without increasing their risk of developing breast cancer. Different estrogenic compounds may exert opposite effects on breast cells. For example, estrogens, such as estradiol (E₂), promote breast cancer; whereas phytoestrogens may actually contribute to the low incidence of breast cancer that is observed in Asia. Although there are substantial laboratory and observational data to support this trend (Kurtzer M. Phytoestrogen supplement use by women. J. Nutr. 2003; 133: 1983S-1986S), to date no randomized controlled studies have documented that phytoestrogens reduce breast cancer risk.

Basic and clinical research has been recently performed using a botanical extract composition, MF101, which is composed of 22 individual plants used in TCM. MF101 is described in Cohen, U.S. patent application Ser. No. 11/277,811; publication No. US 2006/0222721, published Oct. 5, 2006 which is incorporated herein by reference in its entirety. A Phase 1 clinical trial with 22 postmenopausal women found that MF101 was safe for short term use and moderately reduced hot flashes after 30 days of treatment. It was demonstrated that MF101 acts as an ERβ-selective agonist by regulating gene transcription via ERβ pathways (Cvoro et al., Endocrinology 148:538-547 (2007)). It was also shown that MF101 does not stimulate MCF-7 breast cancer cell proliferation or uterine growth in a mouse xenograft model (Cvoro et al., Endocrinology 148:538-547 (2007)). A Phase 2 clinical trial with MF101 for the treatment of hot flashes is underway to further evaluate its safety and efficacy (http://clinicaltrials.gov/show/NCT00119665). These findings suggest that plants used in TCM might be a source for the discovery of estrogen receptor beta (ERβ) subtype selective drugs to safely treat menopausal symptoms. Further, individual compounds have the potential to be safer than the crude herbal formulation since some of the non-therapeutic compounds might elicit adverse affects.

After the Women's Health Initiative found that the risks of hormone therapy (HT) outweigh the benefits, a need for safer drugs to treat menopausal symptoms has emerged. One approach to development of safer alternatives to HT is to isolate and characterize individual chemical entities from known TCM herbal compositions, such as MF101, for use as drugs and compositions for treatment of menopausal symptoms. Individual compounds have the potential to be safer than crude herbal formulations since some of the non-therapeutic compounds might elicit adverse affects. Active compounds can also be synthesized and quantified, allowing for the administration of known amounts and higher doses of the active drug.

### SUMMARY OF THE INVENTION

In one embodiment, the disclosure provides a pharmaceutical composition comprising an isolated and purified compound of formula:

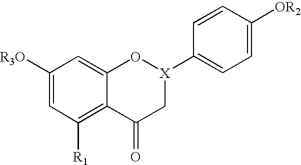

wherein X is an asymmetric carbon atom having an S or R configuration; R₁ is selected from the group consisting of H and OR₄; and R₂, R₃, and R₄ are independently selected from the group consisting of H, and glycoside, glucuronide, acyl, phosphate, phosphonic acid, alkyl phosphonate, sulfate, C₁ to C₆ alkyl, C₃ to C₆ cycloalkyl, aryl, carbonate, and carbamate; each optionally substituted with from one to three groups selected from hydrogen, C₁ to C₆ alkyl, phenyl, benzyl, alkylphenyl, hydroxy, alkoxy, acyloxy, amino, carboxy and alkoxycarbonyl; or a pharmaceutically acceptable salt, or prodrug thereof, a pharmaceutically acceptable salt of said prodrug, and a pharmaceutically acceptable carrier, vehicle, or diluent. In one aspect, X is in the S configuration, and R₁ is H. In another aspect, R₂ and R₃ are selected from H, and optionally substituted glycoside, glucuronide, phosphate, sulfate, acetate, benzoate and carbamate. In a further aspect, R₂ and R₃ are selected from H and glycoside. In a specific aspect, R₂ and R₃ are H, and the compound is of the formula:

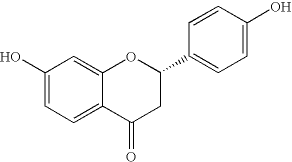

and pharmaceutically acceptable salts thereof.

In another embodiment, the disclosure provides a method of treating one or more menopausal symptoms in a subject in need of such treatment, wherein the method comprises administering an effective amount of the disclosed composition comprising liquiritigenin or a derivative or prodrug thereof. Menopausal symptoms include one or more of hot flashes, sweating secondary to vasomotor instability, hot flashes, fatigue, irritability, insomnia, inability to concentrate, depression, memory loss, headache, anxiety, nervousness, intermittent dizziness, paresthesias, palpitations, tachycardia, nausea, constipation, diarrhea, arthralgia, myalgia, cold hands and feet, weight gain, changes to the genitals, urinary incontinence, vaginal dryness, decreased libido, urinary incontinence, depression loss of pelvic muscle tone, increased low density lipoprotein, increased risk of cardiovascular disease and osteoporosis. In one specific aspect, the menopausal symptom is hot flashes.

In another embodiment, the disclosure provides a method of treating an estrogen receptor beta-mediated disorder in a subject, comprising administering to the subject in need thereof an effective amount of the disclosed composition comprising liquiritigenin or a derivative or prodrug thereof. In one aspect, the estrogen receptor beta-mediated disorder is an estrogen-dependent cancer. In this aspect, the estrogen-dependent cancer is selected from one or more of breast cancer, endometrial cancer, ovarian cancer, uterine adenocarcinoma and vaginal cancer. In another aspect, the estrogen receptor beta-mediated disorder is selected from the group consisting of a disorder of the breast, disorder of the prostate, inflammatory disorder, autoimmune disorder, disorders of the arteries, disorder of the intestine, disorder of the nervous system, disorder of the urinary system, disorder of the ovary, and pain. In another aspect, the disorder of the breast is selected from one or more of benign breast hyperplasia, atypical breast hyperplasia, and fibrocystic breast disorder. In a further aspect, the disorder of the prostate is selected from prostate cancer and benign prostatic hyperplasia. In another aspect, the inflammatory disorder is selected from one or more of Crohn's disease, and colitis. In a further aspect, the autoimmune disorder is selected from rheumatoid arthritis, lupus erythematosis, and Sjogren's syndrome. In another aspect, the disorder of the arteries is selected from one or more of atherosclerosis, peripheral artery disease, coronary stenosis, and coronary restenosis. In a further aspect, the disorder of the intestine is selected from one or more of one or more disorders of the intestine is selected from colon cancer, intestinal cancer, and adenocarcinoma. In another aspect, the disorder of the nervous system is selected from one or more of senile dementia, Alzheimer's disease, menopausal depression, insomnia, menopausal hot flashes, and decreased libido. In a further aspect, the disorder of the urinary system is selected from one or more of dysuria, urinary incontinence, and frequent urination. In another aspect, the disorder of the ovary is selected from one or more of polycystic ovary and unovulation. In a further aspect, the pain is associated with one or more of arthritis, osteoarthritis, and dysmenorrhea.

In another embodiment, the disclosure provides a pharmaceutical composition consisting essentially of a compound of the formula:

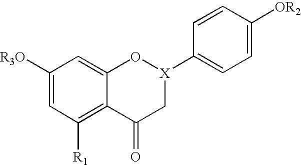

wherein X is an asymmetric carbon atom having an S or R configuration; R₁ is selected from the group consisting of H and OR₄; and R₂, R₃, and R₄ are independently selected from the group consisting of H, and glycoside, glucuronide, acyl, phosphate, phosphonic acid, alkyl phosphonate, sulfate, C₁ to C₆ alkyl, C₃ to C₆ cycloalkyl, aryl, carbonate, and carbamate; each optionally substituted with from one to three groups selected from hydrogen, C₁ to C₆ alkyl, phenyl, benzyl, alkylphenyl, hydroxy, alkoxy, acyloxy, amino, carboxy and alkoxycarbonyl; or a pharmaceutically acceptable salt, or prodrug thereof, a pharmaceutically acceptable salt of said prodrug, and a pharmaceutically acceptable carrier, vehicle, or diluent. In one aspect, X is in the S configuration, and R₁ is H. In another aspect, R₂ and R₃ are selected from H, and optionally substituted glycoside, glucuronide, phosphate, acetate, benzoate and carbamate. In a further aspect, R₂ and R₃ are selected from H and glycoside. In one specific aspect, R₂ and R₃ are H, and the compound is of the formula:

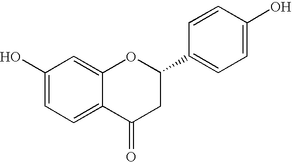

and pharmaceutically acceptable salts thereof.

### BRIEF DESCRIPTION OF THE DRAWINGS

FIG. 1 shows a scheme for chemical synthesis of racemic liquiritigenin.

FIG. 2 shows luciferase activity in (A) U2OS osteosarcoma cells, (B) HeLa cervical cancer cells, and (C) WAR5 prostate cancer cells; which were cotransfected with ERE tkLuc and either ERα or ERβ expression vectors, then treated with various amounts of liquiritigenin for 18 hours.

FIG. 3 shows luciferase activity in U2OS osteosarcoma cells which were transfected with (A) TAT3-luciferase and androgen receptor (AR); (B) MMTV-luciferase and glucocorticoid receptor (GR); (C) TAT3-luciferase and progesterone receptor B(PR); and (D) F2-tkLuc and thyroid hormone receptor β1 (TR); then treated for 18 hour with either (A) 1 nM dihydrotestosterone (DHT), (B) 1 nM dexamethasone (Dex), (C) 1 nM progesterone (Prog), and (D) 10 nM triiodothyronine (T3), respectively, or 2.5 uM liquiritigenin. Each point shown is the average of triplicate determinations±S.E.M.

FIG. 4 shows luciferase activity of U2OS osteosarcoma cells cotransfected with one of (A) CECR6-tk-Luc, (B) NKG2E-tk-Luc, and (C) NKD-tk-Luc and either ERα or ERβ; then treated with various amounts of liquiritigenin for 18 hours.

FIG. 5 shows relative mRNA expression in U2OS cells stably transfected with tetracycline inducible ERα or ERβ, treated with doxycycline for 18 h to induce ER expression, then treated with liquiritigenin for various time intervals. The level of (A) CECR6, (B) NKG2E, and (C) NKD mRNA was measure by real-time PCR, each data point is the average of triplicate determinations+/−S.E.M.

FIG. 6 shows (A) binding of fluorescent-labeled E₂ to purified Erα or ERβ in the absence or presence of increasing amounts of liquiritigenin. (B) shows U2OS-ERβ or U2OS-ERα cells treated with liquiritigenin for various times, then subjected to ChIP assay using antibodies to SRC-2. Real-time PCR was performed to amplify the level of ER regulatory element in (A) CECR6, (B) NKG2E, and (C) NKD genes. Each data point is an average of triplicate determinations+/−S.E.M.

FIG. 7 shows gross morphology in a mouse xenograft models utilizing MCF-7 breast cancer cells grafted under the kidney capsule at the arrows in (A) control, (B) E₂, and (C) liquiritigenin treated mice. Average weights+/−S.E.M. of (D) tumor grafts, and (E) uterine horns from each group (n=5) are shown; * indicates a significant difference between control and drug treatment groups (p<0.05).

### DETAILED DESCRIPTION

The term “menopause” is defined as that period after the cessation of normal ovulation cycles, during which normal menstruation ceases. A decrease in estradiol (E₂) production by the ovaries accompanies menopause. This decrease in E₂ production results in a shift in hormone balance in the body, which often gives rise to a variety of symptoms associated with menopause.

The term “peri-menopause”, which is also known as pre-menopause or the climacteric, is defined as that period prior to menopause during which normal ovulation cycles gradually give way to cessation of menses. As the ovulatory cycles lengthen and become more irregular, the level of E₂ may initially increase, but will eventually drop with the onset of menopause. Menopausal symptoms often accompany the drop in E₂ levels.

The term “menopausal symptoms” is defined as symptoms of one or more of peri-menopause, menopause and post-menopause and include physical symptoms such as hot flashes, and sweating secondary to vasomotor instability. Psychological and emotional symptoms may also accompany onset of climacteric, such as fatigue, irritability, insomnia, inability to concentrate, depression, memory loss, headache, anxiety and nervousness. Additional symptoms can include intermittent dizziness, paresthesias, palpitations and tachycardia as well as nausea, constipation, diarrhea, arthralgia, myalgia, cold hands and feet and weight gain. In addition, changes to the genitals, urinary incontinence, vaginal dryness, loss of pelvic muscle tone, increased low density lipoprotein (LDL), increased risk of cardiovascular disease and osteoporosis increase with onset of menopause.

The term “treatment of menopause” means the alleviation, palliation or prevention of one or more symptoms associated with peri-menopause, menopause or post-menopause, and includes reduction in the severity or frequency of at least one menopausal symptom. The use of “or” as used herein is intended to be conjunctive unless otherwise specified. Thus, treatment also includes reduction of both the severity and frequency of at least one menopausal symptom. In the sense that reduction of the frequency and severity of a symptom may be complete, treatment may also include prevention of the symptom. In this regard, it is noted that treatment of menopause does not include prevention of the natural cessation of menses in the adult female human, although it does include reduction to undetectable levels the frequency and severity of at least one symptom associated with menopause.

The term “menopausal subject” refers to an adult human female who has once attained menarche and who is experiencing peri-menopause, menopause or post-menopause. One of skill in the art of gynecology will be able to identify the diagnostic characteristics of the onset of menopause and identify a subject as being a “menopausal subject” by art-recognized clinical methods.

The term “estrogen” is defined as a class of steroid hormones, produced mainly by the ovaries in women from puberty until the onset of menopause. Estradiol (17 b-Estra-1,3,5(10)-triene-3,17-diol, E₂) is the predominant estrogen hormone produced by the follicular cells of the ovaries. Estradiol acts as a potent non-selective agonist at both estrogen receptor alpha (ERα) and estrogen receptor beta (ERβ) subtypes. Other estrogens include estrone (E₁) and estriol (E₃). Estrogen is important for normal growth and development of women's breast, uterus and ovaries. Estrogen affects a variety of physiological functions in women including body temperature regulation, maintenance of the vaginal lining, and preservation of bone density. In normal women, estrogen production falls sharply upon the onset of menopause, usually at about 50 years of age. The effects of the loss of estrogen production include increased atherosclerotic deposits (leading to greatly increase incidence of heart disease), decreased bone density (osteoporosis), and fluctuations in body temperature among others.

The term “agonist” refers to a chemical substance that binds to a receptor and activates a response in a cell. An ERβ selective agonist is more effective at activation of ERβ than ERα. The term “antagonist” refers to a chemical substance which also binds to a receptor, but fails to activate the response.

The term “estrogen response element” (ERE) is defined as the specific DNA sequences to which both ER-α and ER-β bind with high affinity. The response element is a recognition site for a transcription factor, in this case the transcription factor is the estrogen receptor.

The term “chromatin immunoprecipitation” (ChIP) refers to a procedure used to determine if a given protein binds to or is localized to a specific DNA sequence. The ChIP technique utilizes in vivo cross linking in cells using formaldehyde to bind chromatin-associated proteins to DNA and then isolates these complexes by immunoprecipitation with specific antibodies.

The term “SRC-2” is defined as steroid receptor coactivator-2, and is also known as glucocorticoid receptor interacting protein 1 (GRIP 1), and nuclear receptor coactivator 2 (NCOA2). SRC-2 is a nuclear receptor coregulatory protein which can serve to regulate ER-mediated transcription.

The term “estrogen receptor” (ER) defines a class of nuclear receptors which are ligand-activated nuclear proteins. After binding, the receptor-ligand complex activates gene transcription. There are two types of estrogen receptors: ERα and ERβ. Binding of a ligand (agonist or antagonist) to an ER results in allosteric changes in the receptor. These changes can lead to the dissociation of chaperone proteins and the dimerization of ER. Estrogen receptors α and β can both homodimerize and, less frequently, heterodimerize. The ligand-receptor complex binds to chromatin-organized DNA sequences in the regulatory region of a target gene. ER binding causes a bend in the DNA toward a major groove and facilitates the interactions of key transcriptional components.

The two known estrogen receptors, ERα and ERβ are members of the steroid nuclear receptor super family. ERα was first cloned in 1986, and about 10 years later a second ER was discovered, termed ERβ. ERα contains 595 amino acids, whereas ERβ contains 530 amino acids. Both receptors are modular proteins made up of three distinct domains. The amino-terminus domain (A/B domain) is the least conserved region, exhibiting only a 15% homology between ERα and ERβ. This domain harbors an activation function (AF-1) that can activate gene transcription activation in the absence of estradiol. The central region of ERs contains two zinc finger motifs that bind to an inverted palindromic repeat sequence separated by three nucleotides located in the promoter of target genes. The DNA binding domains (DBD) in ERα. and ERβ are virtually identical, exhibiting 95% homology.

The carboxy-terminus domain contains the ligand binding domain (LBD), which carries out several essential functions. The LBD contains a region that forms a large hydrophobic pocket where estrogenic compounds bind, as well as regions involved in ER dimerization. The LBD also contains a second activation function (AF-2) that interacts with coregulatory proteins. AF-2 is required for both estrogen activation and repression of gene transcription. The LBDs of ERα and ERβ are only about 55% homologous. The differences in the amino acid composition of the ERα and ERβ LBDs may have evolved to create ERs that have distinct transcriptional roles. This would permit ERα. and ERβ to regulate the activity of different genes and to elicit different physiological effects. This notion is supported by studies of ERα and ERβ knockout mice. For example, the ERα knockout mice have primitive mammary and uterine development, whereas the ERβ knockout mice develop normal mammary glands and uterus. These observations demonstrate that only ERα is required for the development of these tissues. Furthermore, ERα. is more effective than ERβ at activating genes, whereas ERβ is more effective than ERα at repressing gene transcription.

Estrogens can activate or repress gene transcription. There are two characterized pathways for activation of gene transcription, the classical ERE (estrogen response element) pathway and the AP-1 (activator protein-1) pathway. There are at least three essential components necessary for estrogens to regulate the transcription of genes: the ERs (ERα and/or ERβ), the promoter element in target genes and coregulatory proteins. The binding of estradiol (E₂) to the ER leads to a conformational change, which results in several key steps that initiate transcriptional pathways. First, the interaction of E₂ with ER leads to the dissociation of chaperone proteins; this exposes the ER's dimerization surface and DNA binding domain. Loss of the chaperone proteins allows the ERs to dimerize and bind to an ERE in the promoter region of a target gene. Second, the binding of E₂ moves helix 12 of the LBD to create a surface that assembles the AF-2 function of the ER. The AF-2 consists of a conserved hydrophobic pocket comprised of helices 3, 5 and 12 of the ER, which together form a binding surface for the p160 class of coactivator proteins (coactivators), such as steroid receptor coactivator-1 (SRC-1) or glucocorticoid receptor interacting protein 1 (GRIP 1). Coactivators (also known as “coregulators”) contain several repeat amino acid motifs comprised of LXXLL, which project into hydrophobic cleft surrounded by the AF-2's helices. The coactivators possess histone acetylase activity. It is thought that gene activation occurs after the ERs and coactivator proteins form a complex on the ERE that causes the acetylation of histone proteins bound to DNA. The acetylation of histones changes the chromatin structure so that the ER/coregulator complex can form a bridge between the ERE and basal transcriptional proteins that are assembled at the TATA box region of the target gene to initiate gene transcription.

The estrogens used in current HT regimens for treatment of the symptoms of peri-menopause, menopause and post-menopause activate both known estrogen receptor subtypes, ERα and ERβ. While the two estrogen receptors (ER), ERα and ERβ share structural domains and similar affinities for estradiol (E₂), many other ligands bind to ERs and act as agonists or antagonists in various tissues. Although the precise roles of both ERs are not known, the specific activation of each subtype results in different biological outcomes. ERα and ERβ knockout mice have different phenotypes (Hewitt et al., Annu Rev Physiol 67:285-308 (2005)). In addition, E₂ activation of ERα versus ERβ results in different gene regulation patterns (Kian et al., Mol Biol Cell 15:1262-1272 (2004)). Estrogen acts as an agonist on ERα and ERβ in all tissue types, which likely explains the beneficial aspects of HT, but this non-selective action also likely causes the adverse side effects unveiled by the WHI. Drugs that selectively activate ERα or ERβ might mimic some of the beneficial effects while avoiding the untoward effects. Since ERα has been shown to cause breast cancer cellular proliferation and ERβ has been demonstrated to be a tumor suppressor (Paruthiyil et al., Cancer Res 64:423-428 (2004), 21. Strom et al., Proc Natl Acad Sci USA 101:1566-1571(2004)). In one embodiment, the disclosure provides compositions comprising ERβ-selective agonists which serve as safer long-term alternative treatment to traditional HT.

Previous results showed that MF101, a botanical extract based upon TCM, which is composed of 22 individual plants, contains ERβ-selective activity (Cvoro et al., Endocrinology 148:538-547 (2007)). Despite the fact that MF101 is comprised of 22 different botanical agents and a multitude of compounds, MF101 exhibits ERP-selectivity and does not exhibit proliferative effects on human breast cancer cells or the mouse uterus (Cvoro et al., Endocrinology 148:538-547 (2007)). The 22 herbs constituting the MF101 were individually screened for estrogenic activity in transfection assays. Among the 22 herbs, Glycyrrhiza uralensis contained high estrogenic activity. Active compounds were isolated from the individual plant components of MF101, including G. uralensis, for further testing.

Activity-guided isolation of the compounds from the G. uralensis was performed using ERE tkLuc and an expression vector for ERβ. These studies resulted in the identification of the flavanone liquiritigenin.

The present disclosure provides compositions comprising compounds that have useful agonist activity with respect to ERβ. The disclosure further provides methods useful for treating estrogen receptor-mediated disorders in mammalian subjects. Thus, the compounds, compositions, and methods described herein have utility in preventing and/or treating a wide variety of estrogen receptor-mediated disorders including, but not limited to, menopausal symptoms, including hot flashes and osteoporosis, as well as breast cancer, ovarian cancer and uterine cancer. In one embodiment, the disclosure provides methods of isolation of an ERβ-selective agonist, liquiritigenin, from the root of G. uralensis.

In another embodiment, methods of synthetic preparation and characterization of liquiritigenin are disclosed.

In a further embodiment, the biological activity of liquiritigenin on estrogen receptors in cells and animal models is disclosed. In one aspect, binding and transcriptional activation of liquiritigenin through the ERs are disclosed.

In another embodiment, the disclosure provides methods of treating menopausal symptoms with compositions comprising liquiritigenin, or derivatives, analogs or prodrugs thereof.

The term “liquiritigenin” is defined as (2S)-7-hydroxy-2-(4-hydroxyphenyl)-2,3-dihydro-4H-1-benzopyran-4-one, alternatively as 4′,7-dihydroxyflavanone, chemical formula C₁₅H₁₂O₄ with molecular weight 256.25, Chemical Abstracts Service Registry Number (CAS RN) 578-86-9. The structure of liquiritigenin (I) is shown below.

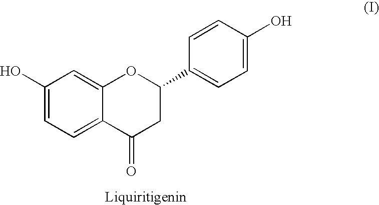

The invention provides compositions and methods for the treatment of menopause, particularly menopausal symptoms such as hot flashes. The compositions of the invention comprise liquiritigenin, or derivatives, or prodrugs thereof. Liquiritigenin, derivatives, analogs or prodrugs are selected from a compound of the formula:

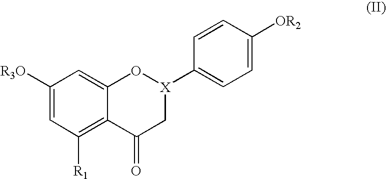

wherein

X is an asymmetric carbon atom having an S or R configuration; R₁ is selected from the group consisting of H and OR₄; and R₂, R₃, and R₄ are independently selected from the group consisting of H, and glycosyl, glucuronyl, acyl, phosphate, phosphonic acid, alkyl phosphonate, sulfate, C₁ to C₆ alkyl, C₃ to C₆ cycloalkyl, aryl, carbonate, and carbamate; each optionally substituted with from one to three groups selected from hydrogen, C₁ to C₆ alkyl, phenyl, benzyl, alkylphenyl, hydroxy, alkoxy, acyloxy, amino, carboxy and alkoxycarbonyl; or a pharmaceutically acceptable salt, or prodrug thereof, a pharmaceutically acceptable salt of said prodrug, and a pharmaceutically acceptable carrier, vehicle, or diluent.

The disclosure also provides pharmaceutically acceptable prodrugs of the compounds of formula I and II. A prodrug is a drug which has been chemically modified and may be biologically inactive at its site of action, but which may be degraded or modified by one or more enzymatic or other in vivo processes to the parent bioactive form.

As used herein, the term “alkyl”, alone or in combinations, means a straight or branched-chain alkyl group containing from one to seven, preferably one to four, carbon atoms such as methyl, ethyl, propyl, isopropyl, butyl, sec-butyl, t-butyl and pentyl. The term “C₁ to C₆ alkyl” is an alkyl limited to one to six carbon atoms.

The term “cycloalkyl”, alone or in combinations, means a three to seven carbon cycloalkyl, for example, cyclopropyl, cyclobutyl, cyclopentyl, cyclohexyl, and cycloheptyl.

The term “alkoxy”, alone or in combinations, is an alkyl covalently bonded by an —O— linkage. Examples of alkoxy groups are methoxy, ethoxy, propoxy, isopropoxy, butoxy and t-butoxy. An alkoxyalkyl is, for example, CH₃(CH₂)ₙ—O—(CH₂)ₘ— wherein m is the from one to seven or preferably one to four and n is 0 to six. The term alkoxycarbonyl is, for example, t-butoxycarbonyl or BOC.

The term “acyl”, alone or in combination, is a moiety is derived from an alkanoic acid containing a maximum of 7, preferably a maximum of 6, carbon atoms (e.g. acetyl, propionyl, butyryl, pentanoyl, pivaloyl) or from an aromatic carboxylic acid (e.g. benzoyl). Also included in acyl is pivaloyl (—(C═O)CH₂CH₂(C═O)CH₃).

The term “aryl”, alone or in combinations means an unsubstituted phenyl group or a phenyl group carrying one or more, preferably one to three, substituents, independently selected from halogen, alkyl, hydroxy, benzyloxy, alkoxy, haloalkyl, nitro, amino, acylamino, monoalkylamino, dialkylamino, alkylthio, alkylsulfinyl, alkylsulfonyl and cyano. The term arylalkyl is preferably benzyl.

The term “glucuronide” represents a glucuronic acid moiety, whose hydroxyl groups are free or O-acetylated, O-methylated, amino, mono, and di-alkylamino substituted, or acylamino substituted.

The term “glycosyl” represents a monosaccharide, disaccharide, polysaccharide, oligosaccharide, aminosaccharide, or deoxysaccharide whose hydroxyl groups are free or O-acetylated, O-methylated, amino, mono, and di-alkylamino substituted, or acylamino substituted. One embodiment of the disclosure provides a compound in which the glycosyl group can be cleaved off by enzymatic hydrolysis.

The term “saccharide” defines a carbohydrate, or sugar, made up of one or more units with the empirical generic formula (CH₂O)ₙ. A saccharide is further classified as a monosaccharide, disaccharide or polysaccharide depending on the number of units or an aminosaccharide if one or more oxygen atoms are replaced by a nitrogen atom. A saccharide may also be classified as a deoxysaccharide if one or more hydroxy groups are replaced by a hydrogen atom. Suitable saccharides include adonitol, arabinose, arabitol, ascorbic acid, chitin, D-cellubiose, 2-deoxy-D-ribose, apiofuranose, dulcitol, (S)-(+)-erythrulose, fructose, fucose, galactose, glucose, inositol, lactose, lactulose, lyxose, maltitol, maltose, maltotriose, mannitol, mannose, melezitose, melibiose, microcrystalline cellulose, palatinose, pentaerythritol, raffinose, rhamnose, ribose, sorbitol, sorbose, starch, sucrose, trehalose, xylitol, xylose and hydrates thereof. Suitable saccharides also include the D and L enantiomers, as well as the alpha and beta anomers of the compounds listed above. Preferred carbohydrates are the simple sugars (e.g., mono- and di-saccharides). A saccharide substituent may be further substituted on any primary or secondary hydroxy group by, for example, an alkyl, alkoxyalkyl, aryl, heteroaryl, ether, ester, acetal, carbonate or carbamate.

The term “monosaccharide” defines a single carbohydrate, or sugar unit. Two families of monosaccharides are aldoses or ketoses. Aldoses have a carbonyl group at the end of the carbon chain as an aldehyde, when the monosaccharide is written in a linear, open-chain formula. If the carbonyl is in any other position in the carbon chain the monosaccharide is a ketone and referred to as a ketose. Three carbon monosaccharides are trioses: glyceraldehydes, an aldose, and dihydroxyacetone, a ketose. Monosaccharides, except for dihydroxyacetone, have one or more asymmetric centers. The prefixes D- or L- refer to the configuration of the carbon atom of the chiral carbon most distant from the carbonyl carbon. Monosaccharides with 4, 5, 6 and 7 carbon atoms in their backbones are termed tetroses, pentoses, hexoses, and heptoses, respectively. Each of these exists in two series: aldotetroses and ketotetroses, aldopentoses and ketopentoses, aldohexoses and ketohexoses, aldoheptoses and ketoheptoses. Tetroses include erythrose and threose. Pentoses include ribose, arabinose, xylose and lyxose. Hexoses include allose, altrose, glucose, mannose, gulose, idose, galactose and talose. Monosaccharides with 5 or more carbons in the backbone usually occur as cyclic, or ring, structures in which the carbonyl carbon has formed a covalent bond with one of the hydroxy groups along the chain. Six-membered monosaccharide ring compounds are termed pyranoses, five-membered monosachharide ring compounds are furanoses. Formation of a six-membered ring results from reaction of aldehydes and alcohols to form hemi-acetals which contain an asymmetric carbon atom. One configuration around the C-1 carbon is described as α- and the other is described as the β-form.

The term “disaccharide” refers to a molecular moiety containing two monosaccharides covalently bound to each other. Disaccharides include maltose [glucose-glucose], lactose [galactose-glucose] and sucrose [fructose-glucose].

The term “polysaccharide” includes multiple monosaccharides units covalently bound to each other. Polysaccharides include starch, hyaluronic acid, amylose, amylopectin, dextran, cyclodextrin and glycogen.

The term “aminosaccharide” refers to a carbohydrate molecule where one or more hydroxy groups are replaced by an amino group. This includes the monosaccharides glucosamine and muramic acid and the polysaccharide chitin. The amino groups may be acetylated to include N-acetyl-D-glucosamine and N-acetyl-D-muramic acid.

The term “deoxysaccharide” refers to a carbohydrate molecule where one or more hydroxy groups are replaced by hydrogen. These include, for example, L-rhamnose (6-deoxy-L-mannose), L-fucose (6-deoxy-L-galactose) and D-fucose (rhodeose).

The phrase “optionally substituted” is used interchangeably with the phrase “substituted or unsubstituted.” Unless otherwise indicated, an optionally substituted group may have a substituent at each substitutable position of the group, and each substitution is independent of any other. Also, combinations of substituents or variables are permissible only if such combinations result in stable compounds. In addition, unless otherwise indicated, functional group radicals are independently selected. Where “optionally substituted” modifies a series of groups separated by commas (e.g., “optionally substituted A, B or C”; or “A, B or C optionally substituted with”), it is intended that each of the groups (e.g., A, B and C) is optionally substituted.

The term “pharmaceutically acceptable salts” includes, but is not limited to, salts well known to those skilled in the art, for example, mono-salts (e.g. alkali metal and ammonium salts) and poly salts (e.g. di- or tri-salts,) of the compounds of the invention. Pharmaceutically acceptable salts of compounds of formula I or II are where, for example, an exchangeable group, such as hydrogen in —OH or —NH— is replaced with a pharmaceutically acceptable cation (e.g. a sodium, potassium, or ammonium ion) and can be conveniently be prepared from a corresponding compound of formula I by, for example, reaction with a suitable base. In cases where compounds are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts. Pharmaceutically acceptable salts may be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example, calcium) salts of carboxylic acids can also be made.

The term “isolated and purified” refers to a compound of the formula I or II which has been obtained by either extractive isolation from a natural source such as a plant species, or chemical synthesis, or a combination thereof; and then purified by means of chromatography, crystallization, distillation, or other means familiar to one skilled in the chemical arts, such that the isolated and purified compound is at least about 90% pure, and preferably at least about 95% pure, as measured by an appropriate analytical chromatographic technique, such as reversed-phase HPLC.

In one embodiment, the disclosure provides a composition comprising a compound of formula (II) wherein the asymmetric carbon has S configuration, R₁ is OH, R₂, R₃ are H, and the composition comprises narigenin (4′,5,7-trihydroxyflavanone; (S)-2,3-dihydro-5,7-dihydroxy-2-(4-hydroxyphenyl)-4H-1-benzopyran-4-one). In another embodiment, the asymmetric carbon has S configuration; R₁, R₃ are H, and R₂ is a glycoside. In a specific aspect, the composition comprises liquiritin. In a further specific embodiment, the composition comprises liquiritigenin-glycoside-apiofuranoside where the asymmetric carbon has S configuration, R₁, R₃ are H, and R₂ is glucoside-apiofuranoside. In a preferred embodiment, the asymmetric carbon atom has S configuration, R₂, R₃, R₄ are H, and the composition comprises liquiritigenin (I).

In one embodiment, derivatives and prodrugs of liquiritigenin are prepared by techniques familiar to one skilled in the art. Synthetic procedures for derivitization of one or more phenolic hydroxy groups of liquiritigenin (I) or a compound of formula (II) are described, for example, in Greene et al., “Protecting Groups in Organic Synthesis”, 2^{nd} Ed. 1991, John Wiley and Sons, New York, pp. 143-170. Benzoate phenolic derivatives may be prepared, for example, by the techniques of Lu et al., Biorg. Med. Chem. Lett. 15: 2607-2609 (2005). Phosphate and phosphate ester prodrugs may be prepared, for example, by the techniques of Simoni et al., J. Med. Chem. 49: 3143-3152 (2006) or Pettit and Lippert, Anti-Cancer Drug Des. 15:203-216 (2000). Mono- and diphosphorylated phenolic derivatives and phosphate esters may be also prepared, for example, by the techniques of Casagrande et al., U.S. Pat. No. 5,073,547. Synthesis of phenolic glycosides may be performed, for example, by the techniques of Fujiwara, Agric. Biol. Chem. 55 (8): 2123-2128 (1991) or Sato et al., Carbohydr. Res., 341(8):964-970 (2006). Phenolic glucuronides may be prepared, for example by the techniques of Roffler et al. U.S. Pat. No. 6,043,367. Carbamate phenolic derivatives may be prepared, for example, by the techniques of Igarahi et al., Chem. Pharm. Bull., 55(2): 328-333 (2007).

In one embodiment, the disclosure provides compositions comprising liquiritigenin, derivatives, or prodrugs. The compositions of the disclosure activate the estrogen response element (ERE) with estrogen receptor beta (ERβ), but not estrogen receptor alpha (ERα), in U2OS osteosarcoma cell assays. As the compositions activate the ERE through interaction with ERβ but not ERα, only the latter of which is associated with adverse effects of estrogen HT, the invention compositions and methods disclosed herein provide an alternative to estrogen hormone therapy for the treatment of menopausal symptoms and are less likely to give rise to conditions identified in the WHI as being associated with estrogen supplementation, such as increased risk of breast cancer.

In one embodiment, liquiritigenin induces only ERβ-specific pathways in transfection assays. In certain aspects, liquiritigenin activates ERE-tk-luciferase, as well as three native ER regulatory elements (NKG2E, CECR6, and NKD) in cells transfected with ERβ but not with ERα. In this aspect, the ERβ-selectivity is also observed with the native NKG2E, CECR6, and NKD genes as demonstrated by the finding that no activation of these genes occurred in the U2OS-ERα cells. In another aspect, the mechanism for the ERβ-selectivity is unlikely related to differences in the binding to ERα and ERβ, because ERβ only has a 20-fold higher affinity for liquiritigenin compared to ERα. In another aspect, ChIP studies showed that liquiritigenin recruits SRC-2 to the NKG2E, CECR6, and NKD genes only in U2OS-ERβ cells, not U2OS-ERα cells. Without being bound by theory, these findings suggest that the selectivity of liquiritigenin is due to the differential recruitment of coactivators to ERβ.

The major problem with HT is not a lack of efficacy, but rather its proliferative effects on breast cancer cells. Therefore, it is essential to rule out a proliferative action for any alternative drug for HT. In one embodiment of the disclosure, liquiritigenin does not stimulate breast cancer tumor formation. In one aspect, in a mouse xenograft model, liquiritigenin does not stimulate breast cancer tumor formation after 30 days of treatment, as compared to therapeutic doses of E₂, which causes the formation of large tumors. In a related aspect, unlike E₂, liquirtigenin does not increase the size of the uterus. In another aspect, liquiritigenin is ERβ-selective in animals, since the proliferative effects on breast and uterine cells are mediated by ERα as demonstrated in the ER knockout mice (Hewitt et al., Annu Rev Physiol 67:285-308 (2005)). The lack of stimulation of breast and uterine cells by liquiritigenin is consistent with the findings that the synthetic ERβ-selective drug, ERB-041 also does not elicit any proliferative effects on mammary and uterine tissue in rats (Harris et al., Endocrinology 144:4241-4249 (2003)). The data with liquiritigenin, MF101 and ERB-041, as well as the findings that ERβ acts as a tumor suppressor in breast cancer cells (Paruthiyil et al., Cancer Res 64:423-428 (2004), 21. Strom et al., Proc Natl Acad Sci USA 101:1566-1571 (2004)), indicate that ERβ-selective agonists will likely have a safer profile than the estrogens currently used in HT that activate both ERα and ERβ.

While plants are known to contain many estrogenic compounds (Oerter et al., J Clin Endocrinol Metab 88:4077-4079 (2003)), their selectivity for the ER subtypes remains largely unstudied. The isoflavone genistein binds better to ERβ than ERα (Kuiper et al., Endocrinology 139:4252-4263 (1998)), and exhibits ERβ-selectivity in transfection studies (An et al., J Biol Chem 276:17808-17814 (2001)).

In one aspect of the disclosure, liquiritigenin is more ERβ selective than genistein. Genistein at 1 μM produced a large activation of ERE-tkLuc (An et al., J Biol Chem 276:17808-17814 (2001)) and activated numerous genes in U2OS-ERα cells. In contrast, in another aspect of the disclosure, liquiritigenin does not activate multiple ER regulatory elements or endogenous genes at the same 1 μM concentration. A related compound, isoliquiritigenin, a trihydroxychalcone, has been shown to activate ERα in MCF-7 cells(Maggiolini et al., J Steroid Biochem Mol Biol 82:315-322 (2002)). Isoliquiritigenin is a non-selective agonist that activates both ERα and ERβ transcriptional pathways. The structural differences between liquiritigenin, genistein, and isoliquiritigenin that result in higher ERβ selectivity with liquiritigenin are currently being investigated.

The crude botanical mixture MF101 is a selective ERβ-agonist, by inducing a functional conformational change in the ERβ receptor that causes the recruitment of coactivators (Cvoro et al., Endocrinology 148:538-547 (2007)). In one embodiment of the disclosure, liquiritigenin is identified as a major active compound from one of the plants in MF101 that is highly selective for ERβ. Human pharmacokinetic studies with MF101 also indicate that liquiritigenin is one the most active ERβ-selective compounds found in plasma. Therefore, in a specific embodiment of the disclosure, liquiritigenin is a viable drug candidate to selectivity activate ERβ.

In one aspect, the disclosure provides a method of treating menopausal symptoms, the method comprising administration of a composition comprising liquiritigenin, or a liquiritigenin derivative, as a ERβ-selective agonist. In a specific aspect, the disclosure provides a method of treating hot flashes, the method comprising administration of a composition comprising liquiritigenin, or a liquiritigenin derivative, as a ERβ-selective agonist. In another specific aspect, the disclosure provides a method of treating osteoporosis, the method comprising administration of a composition comprising liquiritigenin or a liquiritigenin derivative.

In another aspect, the disclosure provides a method of treating an estrogen-dependent cancer, the method comprising administration of a composition comprising liquiritigenin, or a liquiritigenin derivative, as a ERβ-selective agonist. In one aspect, the estrogen-dependent cancer is breast cancer, in another aspect, the estrogen-dependent cancer is endometrial cancer. In yet another aspect, the estrogen-dependent cancer is ovarian cancer. In yet another aspect, the estrogen-dependent cancer is uterine cancer, for example uterine adenocarinoma.

In another aspect, the disclosure provides a method of treating a disorder of the breast, for example, benign breast hyperplasia, atypical breast hyperplasia, and fibrocystic breast disorder, the method comprising administration of a composition comprising liquiritigenin, or a liquiritigenin derivative, as a ERβ-selective agonist.

In a further aspect, the disclosure provides a method of treating one of prostate cancer and benign prostatic hyperplasia, the method comprising administration of a composition comprising liquiritigenin, or a liquiritigenin derivative, as a ERβ-selective agonist.

In another aspect, the disclosure provides a method of treating an inflammatory disorder, for example, Crohn's disease, and colitis, the method comprising administration of a composition comprising liquiritigenin, or a liquiritigenin derivative.

In another aspect, the disclosure provides a method of treating an autoimmune disorder, for example, rheumatoid arthritis, lupus erythematosis, and Sjogren's syndrome, the method comprising administration of a composition comprising liquiritigenin, or a liquiritigenin derivative.

In another aspect, the disclosure provides a method of treating a disorder of the intestine, for example, colon cancer, intestinal cancer, and adenocarcinoma, the method comprising administration of a composition comprising liquiritigenin, or a liquiritigenin derivative.

In another aspect, the disclosure provides a method of treating a disease or disorder of the arteries, for example, atherosclerosis, peripheral artery disease, coronary stenosis, and coronary restenosis, the method comprising administration of a composition comprising liquiritigenin, or a liquiritigenin derivative.

In another aspect, the disclosure provides a method of treating a disorder of the intestine, for example, colon cancer, intestinal cancer, and adenocarcinoma, the method comprising administration of a composition comprising liquiritigenin, or a liquiritigenin derivative.

In another aspect, the disclosure provides a method of treating a disorder of the uterus, for example, uterine adenocarcinoma, uterine fibroids, endometriosis, and dysmenorrhea, the method comprising administration of a composition comprising liquiritigenin, or a liquiritigenin derivative.

In another aspect, the disclosure provides a method of treating pain from, for example, arthritis, osteoarthritis, and dysmenorrhea, the method comprising administration of a composition comprising liquiritigenin, or a liquiritigenin derivative.

In another aspect, the disclosure provides a method of treating a disorder of the urinary system, for example, dysuria, urinary incontinence, and frequent urination, the method comprising administration of a composition comprising liquiritigenin, or a liquiritigenin derivative.

In another aspect, the disclosure provides a method of treating a disorder of the nervous system, for example, senile dementia, Alzheimer's disease, menopausal depression, insomnia, menopausal hot flashes, and decreased libido, the method comprising administration of a composition comprising liquiritigenin, or a liquiritigenin derivative.

In another aspect, the disclosure provides a method of treating a disorder of the vulva or vagina, for example, vaginal dryness, dyspaerunia, and vulvar and vaginal atrophy, the method comprising administration of a composition comprising liquiritigenin, or a liquiritigenin derivative.

In another aspect, the disclosure provides a method of treating a disorder of the ovary, for example, polycystic ovary and unovulation, the method comprising administration of a composition comprising liquiritigenin, or a liquiritigenin derivative.

The disclosure further provides in vivo estrogenic methods of using the disclosed compositions. In general, in vivo methods comprise administering to a subject an amount of liquiritigenin, derivatives or anologs sufficient to bring about an estrogenic effect in the subject. The in vivo methods will give rise to estrogenic ERE-controlled gene activation. Thus, the in vivo methods will give rise to varied positive phenotypic effects in vivo.

The subject may be a mammal, such as a mouse, rat, rabbit, monkey, chimpanzee, dog, cat or a sheep, and is generally female. The subject may also be human, especially a human female. In some embodiments, the subject is a post-menopausal or post-oophorectomic female, and is in need of estrogenic therapy. In such case, the subject may be suffering from climacteric symptoms, such as hot flashes, insomnia, vaginal dryness, decreased libido, urinary incontinence and depression. In other such cases, the subject may be susceptible to, or suffering from, osteoporosis. Suitable in vivo methods include treatment and/or prevention of medical indications that are responsive to estrogen replacement therapy.

Treatment (and its grammatical variants—e.g. treat, to treat, treating, treated, etc.) of a disease, disorder, syndrome, condition or symptom includes those steps that a clinician would take to identify a subject to receive such treatment and to administer a composition of the invention to the subject. Treatment thus includes diagnosis of a disease, syndrome, condition or symptom that is likely to be ameliorated, palliated, improved, eliminated, cured by administering the estrogenic plant extract of the invention to the subject. Treatment also includes the concomitant amelioration, palliation, improvement, elimination, or cure of the disease, disorder, syndrome, condition or symptom. In some embodiments, treatment implies prevention or delay of onset of a disease, disorder, syndrome, condition or symptom m (i.e. prophylaxis), prevention or delay of progression of a disease, disorder, syndrome, condition or symptom, and/or reduction in severity of a disease, disorder, syndrome, condition or symptom. In the case of neoplastic growth in particular, treatment includes palliation, as well as the reversal, halting or delaying of neoplastic growth. In this regard, treatment also includes remission, including complete and partial remission. In the case of climacteric symptoms, treatment includes prevention and palliation of various symptoms.

Prevention (and its grammatical variants) of a disease, disorder, syndrome, condition or symptom includes identifying a subject at risk to develop the disease, disorder, syndrome, condition or symptom, and administering to that subject an amount of the inventive plant extract sufficient to be likely to obviate or delay the onset of said disease, disorder, syndrome, condition or symptom. In some cases, prevention includes identifying a post-menopausal woman who the clinician believes, applying a competent standard of medical care, to be in need of hormone replacement therapy, and administering a composition comprising liquiritigenin, or derivatives or analogs to the woman, whereby one or more climacteric symptoms is blocked or delayed. In some embodiments, prevention of osteoporosis includes identifying a post-menopausal woman who the clinician believes, applying a competent standard of medical care, to be at risk for developing osteoporosis, and administering a composition of the present invention to the woman, whereby the onset of bone loss is blocked or delayed.

Palliation includes reduction in the severity, number and/or frequency of occurrences of an a disease, disorder, syndrome, condition or symptom. Palliation of climacteric symptoms includes reducing the frequency and/or severity of hot flashes, insomnia, incontinence, depression, etc.

Treatment of osteoporosis includes identifying a person, such as a post-menopausal woman, at risk for bone loss, and administering a composition of the present invention to the woman, whereby bone loss is reduced in severity, delayed in onset, or prevented. In some embodiments, treatment of osteoporosis can also include addition of bone mass.

The disclosure further provides methods of obtaining liquiritigenin by extractive isolation from a plant, or by synthetic means. The dislcosure specifically provides a method of extracting liquiritigenin from G. uralensis. The method includes obtaining a quantity of plant matter from a plant of the species G. uralensis, optionally comminuting the plant matter, contacting said plant matter with an extraction medium, and separating the plant matter from the extraction medium.

The magnitude of a prophylactic or therapeutic dose of liquiritigenin derivative, or an analog, derivative or prodrug thereof or a combination thereof, in the acute or chronic management of menopausal symptoms or cancer, e.g. breast cancer, will vary with the severity of the menopausal symptoms or stage of the cancer, such as the solid tumor to be treated, the chemotherapeutic agent(s) or other anti-cancer therapy used, and the route of administration. The dose, and perhaps the dose frequency, will also vary according to the age, body weight, and response of the individual patient. In general, the total daily dose range for liquiritigenin derivative and its analogs, for the conditions described herein, is from about 0.5 mg to about 2500 mg, in single or divided doses. Preferably, a daily dose range should be about 0.5 mg to about 200 mg per day, in single or divided doses, most preferably about 5 to about 50 mg per day. In managing the patient, the therapy should be initiated at a lower dose and increased depending on the patient's global response. It is further recommended that patients over 65 years, and those with impaired renal or hepatic function initially receive lower doses, and that they be titrated based on global response and blood level. It may be necessary to use dosages outside these ranges in some cases. Further, it is noted that the clinician or treating physician will know how and when to interrupt, adjust or terminate therapy in conjunction with individual patient response. The terms “an effective amount” or “an effective sensitizing amount” are encompassed by the above-described dosage amounts and dose frequency schedule.

Any suitable route of administration may be employed for providing the patient with an effective dosage of liquiritigenin or derivative or prodrug (e.g., oral, sublingual, rectal, intravenous, epidural, intrethecal, subcutaneous, transcutaneous, intramuscular, intraperitoneal, intracutaneous, inhalation, transdermal, nasal spray, nasal gel or drop, and the like). While it is possible that, for use in therapy, liquiritigenin derivative or its analogs may be administered as the pure chemicals, as by inhalation of a fine powder via an insufflator, it is preferable to present the active ingredient as a pharmaceutical formulation. The invention thus further provides a pharmaceutical formulation comprising liquiritigenin, a derivative or an analog thereof, together with one or more pharmaceutically acceptable carriers therefore and, optionally, other therapeutic and/or prophylactic ingredients. The carrier(s) must be ‘acceptable’ in the sense of being compatible with the other ingredients of the formulation and not deleterious to the recipient thereof, such as a human patient or domestic animal.

Pharmaceutical formulations include those suitable for oral or parenteral (including intramuscular, subcutaneous and intravenous) administration. Forms suitable for parenteral administration also include forms suitable for administration by inhalation or insufflation or for nasal, or topical (including buccal, rectal, vaginal and sublingual) administration. The formulations may, where appropriate, be conveniently presented in discrete unit dosage forms and may be prepared by any of the methods well known in the art of pharmacy. Such methods include the step of bringing into association the active compound with liquid carriers, solid matrices, semi-solid carriers, finely divided solid carriers or combinations thereof, and then, if necessary, shaping the product into the desired delivery system.

Pharmaceutical formulations suitable for oral administration may be presented as discrete unit dosage forms such as hard or soft gelatin capsules, cachets or tablets each containing a predetermined amount of the active ingredient; as a powder or as granules; as a solution, a suspension or as an emulsion; or in a chewable base such as a synthetic resin or chicle for ingestion of the agent from a chewing gum. The active ingredient may also be presented as a bolus, electuary or paste. Tablets and capsules for oral administration may contain conventional excipients such as binding agents, fillers, lubricants, disintegrants, or wetting agents. The tablets may be coated according to methods well known in the art, i.e., with enteric coatings.

Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The compounds according to the invention may also be formulated for parenteral administration (e.g., by injection, for example, bolus injection or continuous infusion) and may be presented in unit dose form in ampules, pre-filled syringes, small volume infusion containers or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g., sterile, pyrogen-free water, before use.

For topical administration to the epidermis, the compounds may be formulated as ointments, creams or lotions, or as the active ingredient of a transdermal patch. Suitable transdermal delivery systems are disclosed, for example, in A. Fisher et al. (U.S. Pat. No. 4,788,603), or R. Bawa et al. (U.S. Pat. Nos. 4,931,279; 4,668,506 and 4,713,224). Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Formulations suitable for topical administration in the mouth include unit dosage forms such as lozenges comprising active ingredient in a flavored base, usually sucrose and acacia or tragacanth; pastilles comprising the active ingredient in an inert base such as gelatin and glycerin or sucrose and acacia; mucoadherent gels, and mouthwashes comprising the active ingredient in a suitable liquid carrier.

When desired, the above-described formulations can be adapted to give sustained release of the active ingredient employed, e.g., by combination with certain hydrophilic polymer matrices, e.g., comprising natural gels, synthetic polymer gels or mixtures thereof. The polymer matrix can be coated onto, or used to form, a medical prosthesis, such as a stent, valve, shunt, graft, or the like.

Pharmaceutical formulations suitable for rectal administration wherein the carrier is a solid are most preferably presented as unit dose suppositories. Suitable carriers include cocoa butter and other materials commonly used in the art, and the suppositories may be conveniently formed by admixture of the active compound with the softened or melted carrier(s) followed by chilling and shaping in molds.

Formulations suitable for vaginal administration may be presented as pessaries, tampons, creams, gels, pastes, foams or sprays containing, in addition to the active ingredient, such carriers as are known in the art to be appropriate.

For administration by inhalation, the compounds according to the invention are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the compounds according to the invention may take the form of a dry powder composition, for example, a powder mix of the compound and a suitable powder base such as lactose or starch. The powder composition may be presented in unit dosage form in, for example, capsules or cartridges or, e.g., gelatin or blister packs from which the powder may be administered with the aid of an inhalator or insufflator.

For intra-nasal administration, the compounds of the invention may be administered via a liquid spray, such as via a plastic bottle atomizer. Typical of these are the Mistometer.RTM. (Wintrop) and the Medihaler.RTM. (Riker).

For topical administration to the eye, the compounds can be administered as drops, gels (U.S. Pat. No. 4,255,415), gums (see U.S. Pat. No. 4,136,177) or via a prolonged-release ocular insert.

The invention will now be described in greater detail by reference to the following non-limiting examples.

### EXAMPLES

### Example 1

### Isolation and Structural Identification of Liquiritigenin from Glycyrrhiza uralensis

Dry, powdered G. uralensis roots, roots were extracted with 9:1 water-methanol (18 h, constant mixing) at a 10:1 solvent to mass ratio. The filtrate was recovered after suction filtration (Whatman #1 filter), concentrated by rotary evaporation to remove the methanol, and partitioned with an equal volume of ethyl acetate (repeated once). The combined ethyl acetate layers were dried with anhydrous sodium sulfate, concentrated to dryness by rotary evaporation in vacuo, and resuspended in a small volume of ethyl acetate. The sample was loaded onto a fritted glass column packed with silica gel (200-400 mesh, 60 Å) and eluted with a hexane/ethyl acetate gradient, starting with 100% hexane. Liquiritigenin eluted from the silica column with 60-80% ethyl acetate in hexane. The liquiritigenin fractions recovered off the silica column were further purified by preparative reverse phase HPLC (Delta 600 system, Waters Corporation, Milford, Mass.) on a C8 column (SymmetryPrep, 19×150 mm, Waters Corporation) with UV detection (λ=254 nm). A gradient elution from 35-40% acetonitrile in water over 15 min at a flow rate of 12 mL/min was utilized to isolate liquiritigenin at high purity (>95%). Mass spectrometry analysis was performed on a HP 1100 LC/MS (Agilent Technologies, Santa Clara, Calif.), and yielded the expected molecular ion of m/z 255 [M−H]⁻. The ¹H and ¹³C spectra were recorded on a Bruker 500 MHz nuclear magnetic resonance spectrometer (Bruker, Fallanden, Switzerland). NMR spectra were acquired in methanol-d₄ and were consistent with published data for liquiritigenin isolated from Glycyrrhiza species (Fu et al., J Agric Food Chem 53:7408-7414 (2005))

### Example 2

### Synthesis and Characterization of Racemic Liquiritigenin

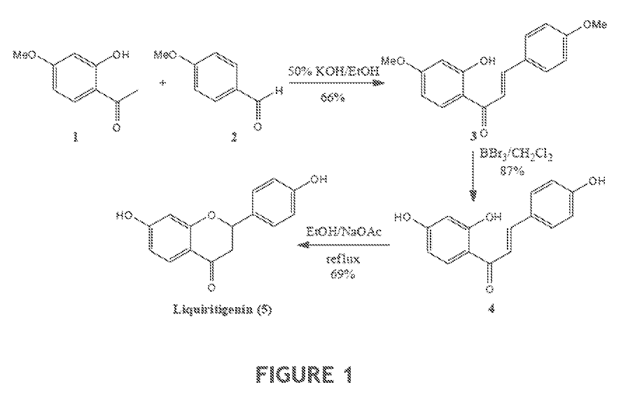

One synthetic scheme for synthesis of racemic liquiritigenin is shown in FIG. 1; synthetic steps and intermediate characterization are described in the following Examples 2a to 2c.

### Example 2a

### 2′-Hydroxy-4,4′-dimethoxychalcone (3)

To a stirred solution of 2-hydroxy-4-methoxy acetophenone (1) (5.24 g, 31.5 mmol) and 4-methoxy benzaldehyde (2) (3.85 mL, 31.7 mmol) in absolute ethanol (100 mL) was added 80 mL of 50% aqueous KOH. The resulting mixture was stirred at room temperature for 48 h. The reaction mixture was acidified at 0° C. with 10% aqueous HCl and then extracted with Et₂O (3×150 mL). The combined ethereal extracts were washed with brine, dried over anhydrous MgSO₄, filtered, and concentrated. The resulting orange yellow solid residue was purified via column chromatography on silica gel (elution with hexane-EtOAc, 8:2) to give an orange yellow solid 5.91 g (20.8 mmol) of 3 (66%). ¹H NMR (CDCl₃): δ 3.86 (6H, s), 6.50 (2H, d, J=10.4 Hz), 6.95 (2H, d, J=8.8 Hz), 7.48 (1H, d, J=15.6 Hz), 7.62 (2H, d, J=9.2 Hz), 7.84 (1H, d, J=9.2 Hz), 7.88 (1H, d, J=15.2 Hz); ¹³C NMR (CDCl₃): δ 55.67, 55.81, 101.27, 107.85. 114.37, 114.69, 118.04, 127.75, 130.59, 131.34, 144.50, 166.26, 166.86, 192.10.

### Example 2b

### 2,4,4′-Trihydroxychalcone (Isoliquiritigenin) (4)

To a well-stirred solution of (3) (8.4 g, 29.5 mmol) in anhydrous CH₂Cl₂ (150 mL) at −78° C. was added drop wise BBr₃ (6.7 mL, 2.5 equiv.). The mixture was stirred at −78° C. for 1 h, then slowly warmed to room temperature and stirred for an additional 24 h. The reaction was quenched by the addition of H₂O (15 mL). The layers were separated and the aqueous layer was extracted with EtOAc and 5% MeOH. The combined organic layers were washed with H₂O and brine, dried over anhydrous MgSO₄, and evaporated under reduced pressure. The resulting residue was purified by silica gel column chromatography eluting with hexane-EtOAc (8:2) to give 4 (6.58 g, 25.7 mmol, 87%) as an orange yellow solid. ¹H NMR (CDCl₃+CD₃OD): δ 6.38 (2H, d, J=2.0 Hz), 6.45 (1H, d, J=8.8 Hz), 6.88 (2H, d, J=8.4 Hz), 7.47 (1H, d, J=15.6 Hz), 7.56 (1H, d, J=8.4 Hz), 7.81 (1H, d, J=10.0 Hz), 7.83 (1H, d, J=8.0 Hz), ¹³C NMR (CDCl₃+CD₃OD): δ 103.11, 108.30.113.59, 115.97, 117.12, 126.50, 130.61, 131.87, 144.61, 159.83, 164.68, 165.84.

### Example 2c

### Liquiritigenin (5)

To a stirred solution of 4 (3.5 g, 13.6 mmol) in EtOH (60 mL) were added NaOAc (5.5 g, 67 mmol) and water (200 μL). The mixture was heated at reflux for 36 h. After the mixture was cooled to ambient temperature, H₂O was added and the mixture was extracted with Et₂O (3×100 mL). The combined organic layers were washed with H₂O and brine, dried over anhydrous MgSO₄, and evaporated under reduced pressure. The residue was purified by silica gel column chromatography eluting with hexane-EtOAc (9:1.1:1) to give racemic 5 (2.46 g, 9.6 mmol, 69%) as a white solid. ₁H NMR (CDCl₃+CD₃0D): δ 2.76-2.71 (1H, dd, J=16.8, 2.8 Hz), 3.09, 3.04 (1H, dd, J=13.2, 2.8 Hz), 5.38 (1H, d, J=13.2 Hz), 6.41 (2H, d, J=2.4 Hz), 6.54-6.52 (1H, dd, J=8.8, 2.4 Hz), 6.89 (2H, d, J=8.4 Hz), 7.33 (2H, d, J=8.4 Hz), 7.79 (1H, d, J=8.8 Hz), ¹³C NMR (CDCl₃+CD₃0D): δ 44.01, 79.80, 103.13, 111.03. 113.99, 115.63, 127.99, 129.13, 129.84, 157.48, 164.15, 165.19, 192.36.

### Example 3

### Cell Culture, Transfection, and Luciferase Assays

U2OS osteosarcoma cells, MCF-7 human breast cancer cells and HeLa human cervical cancer cells were obtained from the cell culture facility at the University of California, San Francisco. The MCF-7 cell line is a well established model for the study of E₂-induced human breast cancer cell growth and was thus selected for this study (35). WAR5 prostate cancer cells were prepared as previously described (Ricke et al., Int J Cancer 118:2123-2131(2006)). All cell lines were maintained and subcultured as previously described (An et al., Proc Natl Acad Sci USA 96:15161-15166 (1999)). Transfections were carried out with a Bio-Rad gene pulser. Cells were electroporated and cotransfected with 3 μg of one of ERE or CECR6 or NKD, or NKG2 thymidine kinase (tk)-Luciferase reporter vectors along with 1 μg of one of ERα or ERβ expression vectors. After electroporation, the cells were plated and treated with E₂ or liquiritigenin for about 24 h. Cells were then solubilized and luciferase activity was determined (Promega, Madison, Wis).

### Example 3a

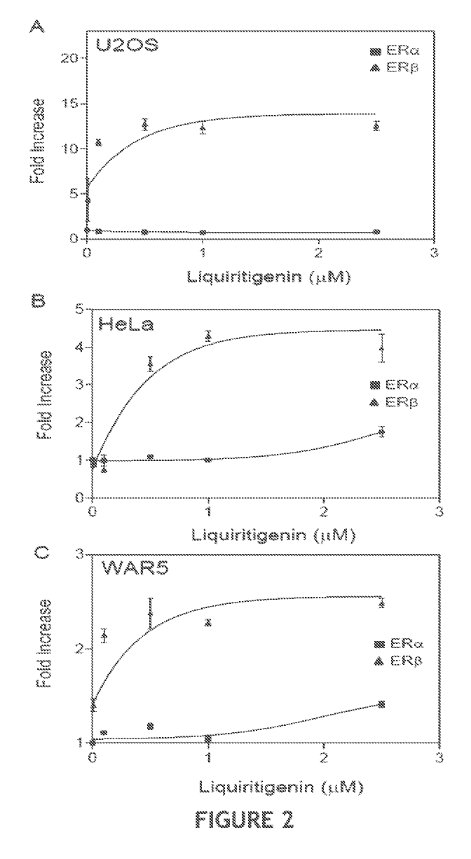

To assess the relative activity of liquiritigenin via ERα or ERβ, transfection assays were used with increasing concentrations of liquiritigenin. ERE tkLuc was cotransfected into cells with expression vectors for ERα or ERβ. After transfection, the cells were treated for 18 h with increasing amounts of liquiritigenin and luciferase activity was measured. Results are shown in FIG. 2. Liquiritigenin produced a dose-response activation of luciferase in the U2OS cells transfected with ERA, but not ERα (FIG. 2A). The activation first occurred at 1 nM and the maximal activation was observed at 500 nM. The ERβ-selectivity of liquiritigenin was also observed in HeLa cells (FIG. 2B) and the prostate cancer WAR5 cell line (FIG. 2C). Therefore, liquiritigenin selectively activates the ERE with ERβ in U2OS osteosarcoma, HeLa cervical and WAR5 prostate cancer cell lines and liquiritigenin selectively activates ERβ transcriptional pathways in multiple cell lines.

### Example 3b

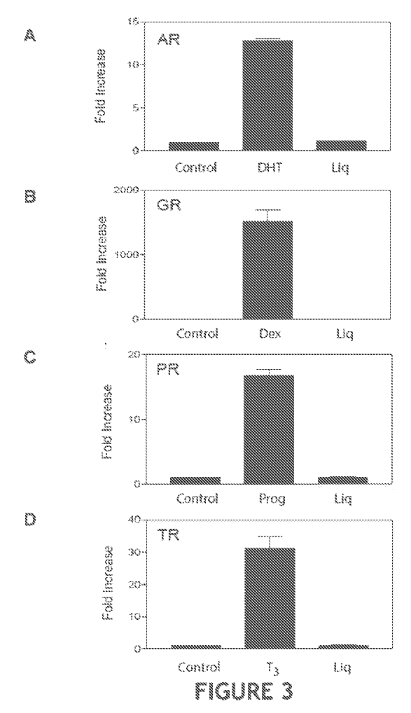

U2OS osteosarcoma cells were transfected with TAT3-luciferase and androgen receptor (AR) (FIG. 3A), MMTV-luciferase and glucocorticoid receptor (GR) (FIG. 3B), TAT3-luciferase and progesterone receptor B (PR) (FIG. 3C), or F2-tkLuc and thyroid hormone receptor β1 (TR) (FIG. 3D). The cells were treated for 18 h with 1 nM dihydrotestosterone (DHT), or 1 nM dexamethasone (Dex), or 1 nM progesterone (Prog), or 10 nM triiodothyronine (T₃) (FIGS. 3D, E, F, and G, respectively) or 2.5 μM liquiritigenin (Liq). Each data point is the average of triplicate determinations±S.E.M. Results are shown in FIGS. 3A to 3D. Liquiritigenin did not activate other nuclear receptors including the androgen receptor (AR), glucocorticoid receptor (GR), progesterone receptor B (PR) and thyroid hormone receptor (TR) in transfection assays.

### Example 4

### Real-Time PCR

U2OS cells expressing a tetracycline-inducible ERα or ERβ cDNA were prepared as previously described (Kian et al., Mol Biol Cell 15:1262-1272 (2004). Cells were treated with doxycycline (100 ng/ml) for 16-20 hours and then with E₂ or liquiritigenin for 3 hours. Total RNA was isolated using Trizol (Invitrogen Life Technologies, Carlsbad, Calif.) and reverse transcription (RT) reactions were performed using iScript cDNA Synthesis Kit (Bio-Rad, Hercules, Calif.). Real-time quantitative PCR was performed using SYBR Green Supermix with an iCycler thermal cycler (Bio-Rad). The following primers were used.

| | | |
|---|---|---|
| NKD | Forward | 5′-CAGCCAGAGCAAGAGGAGCGTC-3′ |
| | Reverse | 5′-CCGGCGAGATCTAAGTAGTGGT-3′ |
| NKG2E | Forward | 5′-GCCAGCATTTTACCTTCCTCAT-3′ |
| | Reverse | 5′-AACATGATGAAACCCCGTCTAA-3′ |
| CECR6 | Forward | 5′-ACAGTCGGTGTGGAATGTC-3′ |
| | Reverse | 5′-AGAAGGGAGAAGGGGAAACA-3′ |

### Example 4A

### Liquiritigenin Activation of Transcription of Native ER Regulatory Elements and Genes Through ERβ

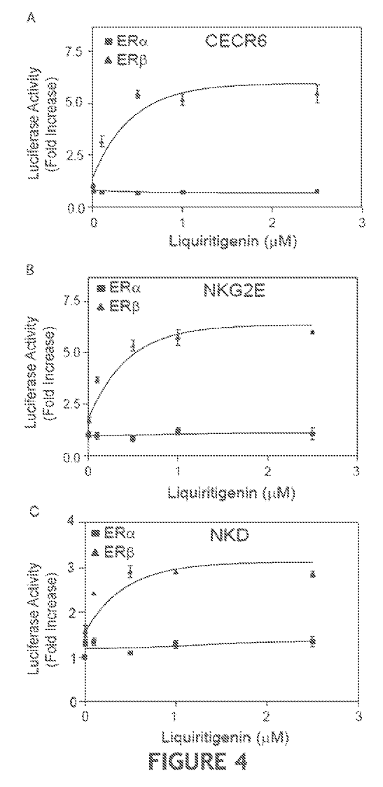

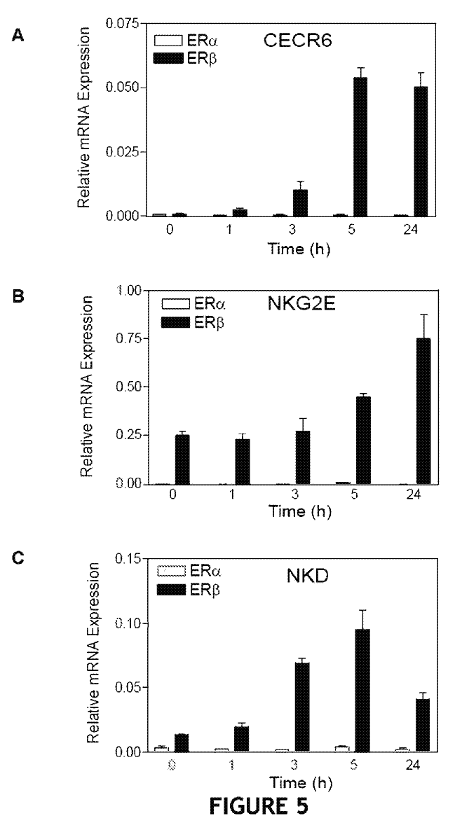

In addition to the traditional ERE, it is important to determine if liquiritigenin selectivity activates ERβ in elements derived from native ER target genes. ER regulatory elements from the cat eye syndrome chromosome region candidate 6 (CECR6), killer cell lectin-like receptor (NKG2E) and the naked cuticule homolog (NKD) genes that are activated by E₂ ( Levy et al., Endocrinology doi:10.1210/en.2006-1632 (2007)) were used. CECR6-tk-Luc (A), NKG2E-tk-Luc (B), and NKD-tk-Luc (C) were transfected into U2OS cells with expression vectors for human ERα or ERβ. After transfection, the cells were treated for 18 h with increasing amounts of liquiritigenin and luciferase activity was measured. Results are shown in FIG. 4. Liquiritigenin produced a dose-dependent activation of CECR6, NKG2E, and NKD with ERβ but not with ERα (FIGS. 4A, 4B, 4C, respectively). The cells were then treated for increasing times with liquiritigenin. The level of CECR6, NKG2E, and NKD mRNAs was measured by real-time PCR; results are shown in FIG. 5A to C, respectively. Each data point is the average of triplicate determinations±S.E.M. Liquiritigenin produced a time-dependent increase in CECR6 (FIG. 5A), NKG2E (FIG. 5B) and NKD (FIG. 5C) mRNA by real-time PCR in the U2OS-ERβ cells, but not the U2OS-ERα cells. These results demonstrate that liquiritigenin is an ERβ-selective agonist with multiple ER regulatory elements and native target genes.

### Example 5

### ER Binding Assays

The relative binding affinity of liquiritigenin to pure full-length ERα and ERβ was determined using ERα and ERβ competitor assay kits, according to the manufacturer's instructions (Invitrogen Life Technologies, Carlsbad, Calif.). Fluorescence polarization of the fluorophore-tagged estrogen bound to ERα and ERβ in the presence of increasing amounts of competitor ligand or extract was determined (10 readings per well; 0.02 millisecond integration time; G factor=1.1087) using the GENios Pro microplate reader (Tecan Systems Inc., San Jose, Calif.) with fluorescein excitation (485 nM) and emission (530 nM) filters. Each liquiritigenin dose was performed in triplicate and the relative error was determined by calculating the standard error of three values from the mean.

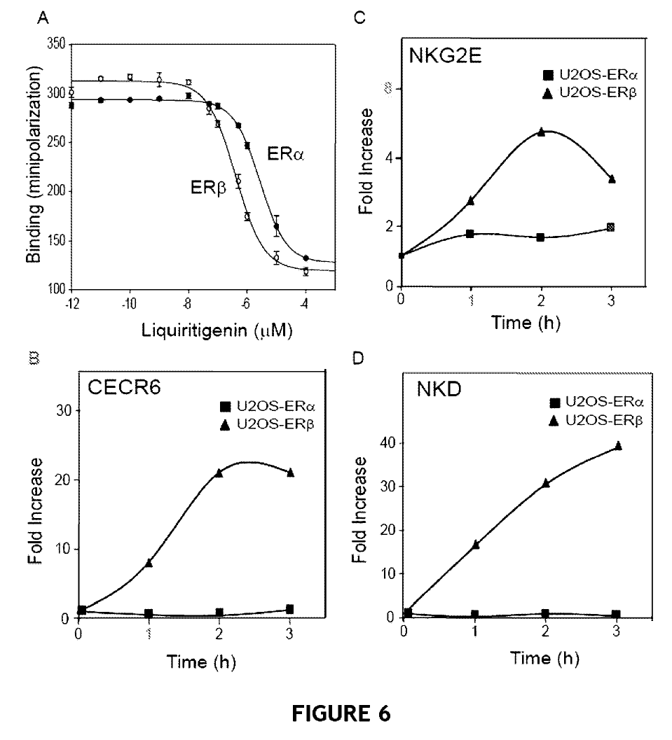

Estrogen receptor ligands have been shown to have different affinities for ERα or ERβ. For example, E₂ binds with equal affinity to both ERα and ERβ, whereas some phytoestrogens such as the isoflavone genistein bind with a higher affinity to ERβ compared to ERα (Barkhem et al., Mol Pharmacol 54:105-112 (1998), Kuiper et al., Endocrinology 139:4252-4263 (1998)). One of the possible mechanisms for the ERβ-selectivity of liquiritigenin is that it binds with higher affinity to ERβ than to ERα. However, competition binding curves show that ERβ only has only about a 20-fold greater affinity for liquiritigenin compared to ERα (FIG. 6A), which is not likely sufficient to explain the differences in transcriptional regulation. A more plausible explanation for the ERβ-selectivity is that liquiritigenin recruits coactivators only to ERβ. To test this hypothesis, U2OS-ERα and U2OS-ERβ cells were incubated with liquiritigenin for increasing times and then ChIP was done with an antibody to the coactivator SRC-2. Focus was placed on SRC-2 because it was previously shown that E₂ recruited only SRC-2 to multiple ER regulatory elements, such as NKG2E, CECR6 and NKD genes (Levy et al., Endocrinology doi:10.1210/en.2006-1632 (2007)).

### Example 6

### Chromatin Immunoprecipitation (ChIP)

Following treatment with liquiritigenin or E₂, stably transfected U2OS-ERα and U2OS-ERβ cells were crosslinked with 1% formaldehyde and ChIP was done as previously described (Cvoro et al., Mol Cell 21:555-564 (2006)).

After cells were treated and crosslinked, they were washed, collected, and lysed. Immunoprecipitations were performed overnight at 4° C. with anti-SRC-2 (ab9261, Abcam, Cambridge, Mass.) antibodies. DNA fragments were purified (QIAquick PCR Purification Kit, Qiagen, Valencia, Calif.) and PCR-amplified. The primers used for ChIP are:

| | | |
|---|---|---|
| CECR6 | Forward | 5′-TGATAAATGCTAGTGAGGTGCC-3′ |
| | Reverse | 5′-AGAACCGCCTGCTCCTAACAAT-3′ |
| NKD | Forward | 5′-GGGTCAGGACGAGTGTTTTCTT-3′ |
| | Reverse | 5′-ACCCCGGACCAAATTTCAGTTA-3′ |
| NKG2E | Forward | 5′-AGCCACCCAAAGTCTCCTAT-3′ |
| | Reverse | 5′-TTCAGTGGAGAGGTCAGGTT-3′. |

PCR reactions for non-immune assays served as negative controls. ChIP results are shown in FIG. 6. Liquiritigenin caused the recruitment of SRC-2 to the CECR6 (FIG. 6B), NKG2E (FIG. 6C), and NKD (FIG. 6D) genes in the U2OS-ERβ cells, but not the U2OS-ERα cells. These results demonstrate that liquiritigenin acts as an ERβ-selective agonist because it only recruits coactivators to ERβ.

### Example 6

### Xenograft Studies in Nude Mice

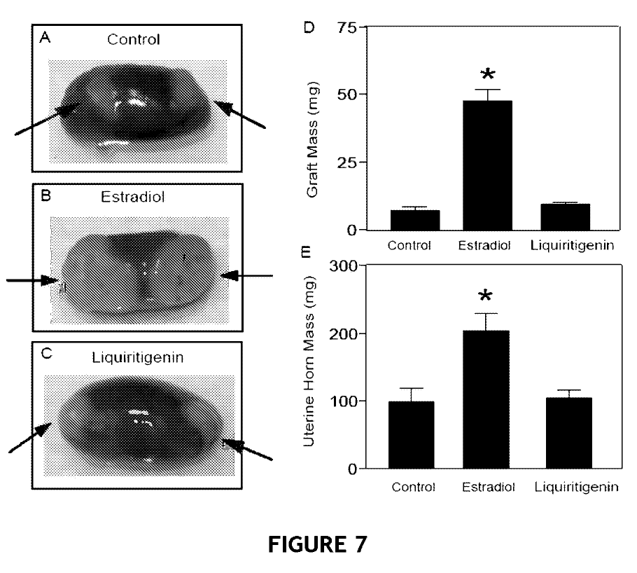

MCF-7 (250,000) cells were aggregated in suspension and then resuspended in 200 μL neutralized collagen, as previously described (Parmar et al., Endocrinology 143:4886-4896 (2002). The cells were then grafted under the kidney capsule of nude mice as described and illustrated in detail at: http://mammary.nih.gov/tools/mousework/Cunha001/index.html. Five mice per group were treated with a continuous infusion using osmotic pumps (Alzet, Cupertino, Calif.) containing vehicle, E₂ (0.4 mg) or liquiritigenin (2 mg) that infused 2.5 μl/h for 1 month. After one month of treatment, the tumors and uteri were removed and analyzed. These animal studies were carried out with approval from the University of California, San Francisco Committee on Animal Research. Results are shown in FIG. 7.

The major concern with estrogens for menopausal symptoms is the proliferation of breast and endometrial cells causing an increased risk for breast and uterine cancer. To determine if liquiritigenin has a proliferative effect on breast cancer and endometrial cells, MCF-7 breast cancer cells were grafted under the kidney capsule of nude mice. Using a subcutaneous osmotic pump designed to deliver a steady dose of drug, the mice were treated for 30 days with vehicle, E₂, or liquiritigenin. Large tumors developed in the mice treated with E₂ (FIG. 7B), while there was essentially no tumor growth in the mice treated with vehicle (FIG. 7A) or liquiritigenin (FIG. 7C). There were no differences in the weight of the tumors in mice treated with liquiritigenin compared to the control mice (FIG. 7D). In addition, after 30 days of treatment, liquiritigenin did not increase uterine horn mass, whereas E₂ did (FIG. 7E). In mouse xenograft models, liquiritigenin does not have proliferative effects on breast cancer cells or on the uterus.

All publications and patent applications mentioned in this specification are herein incorporated by reference to the same extent as if each individual publication or patent application was specifically and individually indicated to be incorporated by reference.

We claim:

1. A pharmaceutical composition comprising an isolated and purified compound of formula: 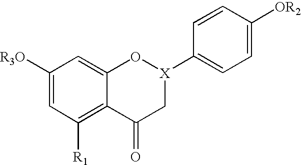
wherein
X is an asymmetric carbon atom having an S or R configuration;
R₁ is selected from the group consisting of H and OR₄; and
R₂, R₃, and R₄ are independently selected from the group consisting of H, and glycoside, glucuronide, acyl, phosphate, phosphonic acid, alkyl phosphonate, sulfate, C₁ to C₆ alkyl, C₃ to C₆ cycloalkyl, aryl, carbonate, and carbamate; each optionally substituted with from one to three groups selected from hydrogen, C₁ to C₆ alkyl, phenyl, benzyl, alkylphenyl, hydroxy, alkoxy, acyloxy, amino, carboxy and alkoxycarbonyl; or
a pharmaceutically acceptable salt, or prodrug thereof, a pharmaceutically acceptable salt of said prodrug, and
a pharmaceutically acceptable carrier, vehicle, or diluent.

2. The composition of claim 1, wherein X is in the S configuration, and R₁ is H.

3. The composition of claim 2, wherein R₂ and R₃ are selected from H, and optionally substituted glycoside, glucuronide, phosphate, sulfate, acetate, benzoate and carbamate.

4. The composition of claim 3, wherein R₂ and R₃ are selected from H and glycoside.

5. The composition of claim 4, wherein R₂ and R₃ are H, and the compound is of the formula: 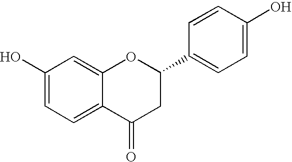
and pharmaceutically acceptable salts thereof.

6. A method of treating one or more menopausal symptoms in a subject in need of such treatment, wherein the method comprises administering a composition comprising an effective amount of a compound of the formula: 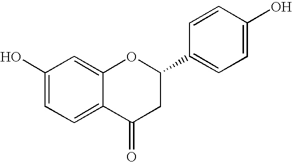
or a pharmaceutically acceptable salt thereof.

7. The method of claim 6, wherein the one or more menopausal symptoms are selected from the group consisting of hot flashes, sweating secondary to vasomotor instability, hot flashes, fatigue, irritability, insomnia, inability to concentrate, depression, memory loss, headache, anxiety, nervousness, intermittent dizziness, paresthesias, palpitations, tachycardia, nausea, constipation, diarrhea, arthralgia, myalgia, cold hands and feet, weight gain, changes to the genitals, urinary incontinence, vaginal dryness, decreased libido, urinary incontinence, depression loss of pelvic muscle tone, increased low density lipoprotein, increased risk of cardiovascular disease and osteoporosis.

8. The method of claim 7 wherein the menopausal symptom is hot flashes.

9. A method of treating an estrogen receptor beta-mediated disorder in a subject, comprising administering to a subject in need thereof a composition comprising an effective amount of a compound of the formula: 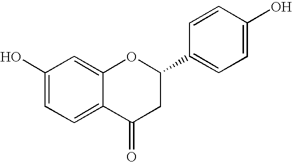
or a pharmaceutically acceptable salt thereof.

10. The method of claim 9, wherein the estrogen receptor beta-mediated disorder is an estrogen-dependent cancer.

11. The method of claim 10, wherein the estrogen-dependent cancer is selected from one or more of breast cancer, endometrial cancer, ovarian cancer, uterine adenocarcinoma and vaginal cancer.

12. The method of claim 9, wherein the estrogen receptor beta-mediated disorder is selected from the group consisting of a disorder of the breast, disorder of the prostate, inflammatory disorder, autoimmune disorder, disorders of the arteries, disorder of the intestine, disorder of the nervous system, disorder of the urinary system, disorder of the ovary, and pain.

13. The method of claim 12, wherein the disorder of the breast is selected from one or more of benign breast hyperplasia, atypical breast hyperplasia, and fibrocystic breast disorder.

14. The method of claim 12, wherein the disorder of the prostate is selected from prostate cancer and benign prostatic hyperplasia.

15. The method of claim 12, wherein the inflammatory disorder is selected from one or more of Crohn's disease, and colitis.

16. The method of claim 12, wherein the autoimmune disorder is selected from rheumatoid arthritis, lupus erythematosis, and Sjogren's syndrome.

17. The method of claim 12, wherein the disorder of the arteries is selected from one or more of atherosclerosis, peripheral artery disease, coronary stenosis, and coronary restenosis.

18. The method of claim 12, wherein the disorder of the intestine is selected from one or more of one or more disorders of the intestine is selected from colon cancer, intestinal cancer, and adenocarcinoma.

19. The method of claim 12, wherein the disorder of the nervous system is selected from one or more of senile dementia, Alzheimer's disease, menopausal depression, insomnia, menopausal hot flashes, and decreased libido.

20. The method of claim 12, wherein the disorder of the urinary system is selected from one or more of dysuria, urinary incontinence, and frequent urination.

21. The method of claim 12 wherein the disorder of the ovary is selected from one or more of polycystic ovary and unovulation.

22. The method of claim 12, wherein the pain is associated with one or more of arthritis, osteoarthritis, and dysmenorrhea.

23-27. (canceled)
